(19) United States
(12) Patent Application Publication
(10) Pub. No.: US 2003/0153897 A1
(43) Pub. Date: Aug. 14, 2003

(54) Closed system drainage and infusion connector valve

(76) Inventor: Ronald D. Russo, Barrington, RI (US)

Correspondence Address: Robert J Doherty Esq., 10 George St., Barrington, RI, 02806, US

(21) Appl. No.: 10/346,655

(22) Filed: Jan. 17, 2003

Related U.S. Application Data
(60) Provisional application No. 60/355,984, filed on Feb. 12, 2002.

(51) Int. Cl. A61M 25/16

(52) U.S. Cl. 604/537; 604/284

(57) ABSTRACT

A convenient, compact and truly closed system drainage and infusion connector valve is disclosed that can be used in conjunction with a wide variety of medical catheters and tubes. The device is designed to take the place of open style adapters and connectors. It has a simple On-Off handle for fluid control and a separately functioning syringe port seal. The device provides in-line irrigation, medication delivery, or syringe decompression/aspiration without disconnect, remaining closed to atmosphere at all times. This closed system protects the clinician from potentially infectious splashback events and the patient from external contamination during suction and drainage, enteral feeding, irrigation/lavage, and medication delivery.

### RELATED APPLICATION

The inventor claims the full benefit of the following U.S. Provisional Patent Application: “Closed System Drainage And Infusion Connector Valve”., filed Feb. 12, 2002, application No. 60/355,984.

### BACKGROUND OF THE INVENTION

Various types of drainage, infusion, and enteral delivery tubes are commonly used in medical practice. Typical of these tubes are nasogastric tubes, Salem sump tubes, Levin tubes, naso decompression tubes, PEG tubes, and balloon replacement gastrostomy tubes.

The nasogastric type tubes are placed nasally down into the stomach or duodenum for drainage or decompression or for the delivery of enteral nutrition. The PEG and gastrostomy type tubes are placed through the abdomen directly into the stomach or duodenum.

For drainage, these tubes use a connector which attaches to the tube at one end and connects to a suction tubing line and in turn to a drainage receptacle or suction pump delivering a regulated source of suction. Sometimes these suction apparatus are portable and sometimes they are permanently fixed at bedside.

Often it is necessary to turn off the suction or delivery set in order to administer liquid or dissolved medication. This often requires disconnection of the system which is awkward or cumbersome. In order to avoid disconnection, a prior art invention was conceived called commercially the “Lopez Valve” which is U.S. Pat. No. 4,895,562 issued January 1990 and similar U.S. Pat. No. 4,790,832 issued December 1988. The “Lopez Valve” uses a 4-way stopcock arrangement clearly depicted in FIGS. 3,4,5,6 of the '562 patent and fully described in that disclosure.

In one of the 4-way positions, the drainage line could be shut off and a piston irrigation syringe attached to the 4-way valve to deliver liquid medications. This arrangement eliminated the need to disconnect the suction tubing line or delivery set from the indwelling tube. While the “Lopez Valve” of the '562 patent was an advancement, it still has several deficiencies. The 4-way stopcock is somewhat confusing to find the right position. If the stopcock handle is left in the syringe infusion mode, stomach contents which are pressurized can spew out the syringe port. In summary, the “Lopez Valve” is dependent upon the user to a great extent in order to function and is not a truly closed system since the syringe port can easily be left open to atmosphere. Further, the 4-way valve has a small passageway wherein infused dissolved solid and crushed medication can easily become clogged in the “Lopez Valve”. Spattering or spewing of potentially infectious stomach contents out the syringe port of the “Lopez Valve” can also be a problem when the syringe is disconnected and if the valve is not immediately closed prior to disconnection of the syringe. The syringe port on the “Lopez Valve” also sticks out the side of the 4-way handle making it awkward and prone to catching on the patient's garment or bedclothes. Most recently, tethered caps have been added to the commercial “Lopez Valve” product, but this has increased confusion in using the device and added bulk to the overall size of the device. In addition, U.S. Pat. Nos. 6,240,960 issued June 2001 and 6,481,462 issued November 2002 to Fillmore describe noncommercial single function valve constructions similar to but more complex than these of Lopez.

### SUMMARY OF THE INVENTION

The difficulties and problems discussed above have been obviated by the present invention which is a truly closed system connector valve and much simpler and easier to use. The present connector invention comprises four components easily assembled into a unitized closed system drainage and infusion connector valve which can attach to a wide variety of catheters and tubes. It comprises a simple On-Off 2-way turn handle valve with a separate functioning built in syringe port. In essence, the syringe port is not part of the On-Off valve and functions automatically to open upon insertion of the syringe tip and to automatically close upon removal of the syringe tip. In summary, the connector valve remains closed to atmosphere at all times.

Splattering of stomach contents is eliminated and dissolved medications can be delivered without clogging. A resilient adapter can be added to the proximal end of the connector valve permitting connection to all enteral delivery sets whether pump or gravity as well as bolus syringe feeding.

The closed system connector valve is streamlined, easy to use and understand, and inexpensive to manufacture.

It is therefore a primary object to provide a closed system drainage and infusion connector valve which is truly a closed system device.

It is another object to provide a connector valve which separates the function of On-Off and syringe infusion.

It is another object to provide a connector valve which prevents splashback through the syringe port.

It is another object to provide a streamlined in-line design connector valve which is easy to use as a closed system adapter for enteral feeding tubes.

Other objects will become readily apparent upon review of the drawings and detailed description of the invention.

### BRIEF DESCRIPTION OF THE DRAWINGS

FIG. 1 is a top view of the prior art “Lopez Valve” in its commercial form as depicted in U.S. Pat. No. 4,895,562.

FIG. 2 is at top view of the closed system drainage and infusion connector valve of the present invention.

FIG. 3 is a top view of the On-Off handle of the closed system drainage and infusion connector valve.

FIG. 4 is a cross sectional view of the connector valve shown in its normal mode of operation with the handle positioned in the on or open position and the syringe port seal in its normally closed sealed position.

FIG. 5 is a cross sectional view of the connector valve in its valve turned to the off or closed position and insertion of a piston syringe into the syringe port seal.

FIG. 6 is a cross sectional view of the connector valve with a resilient adapter added to its proximal end.

FIG. 7 is a top view of the connector valve as part of the Directions For Use in its feeding procedure mode.

FIG. 8 is a top view of the connector valve as part of the Directions For Use in its drainage and suction procedure mode.

FIG. 9 is a top view of the connector valve as part of the Directions For Use in its irrigation/lavage and medication delivery procedure mode.

### DETAILED DESCRIPTION OF THE INVENTION

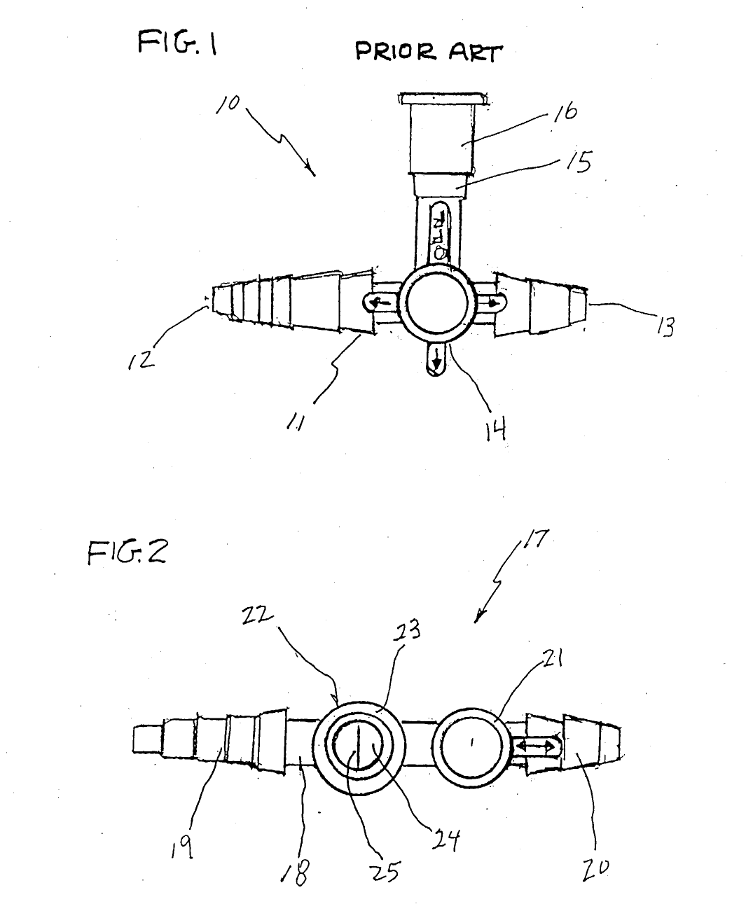

FIG. 1 depicts prior art “Lopez Valve” 10 having an injection molded body 11 with a frontal stepped tubing connector portion 12 and a rearward suction connector 13. Fitted into body 11 is 4-way stopcock 14 having side syringe port 15 as part of the 4-way stopcock. Cap 16 closes off syringe port 15. As can be seen, when 4-way stopcock 14 is in an open positioned 15, stomach contents can exit out syringe port 15 at any time the syringe is not connected to port 15. Likewise, if separate loose cap 16 is lost and the valve 10 is inadvertently turned to open the syringe port 15, stomach contents can again exit out port 15. These above-described drawbacks of the “Lopez Valve” present significant risks to caregivers utilizing this device and are eliminated by the construction of the present invention as hereinafter described.

FIG. 2 is a top view of closed system connector valve 17 preferably having an injection molded one-piece clear polycarbonate body 18. Body 18 has a built-in distal gradually increasing in diameter stepped barbed connector 19 which can be attached to a wide variety of indwelling tubes or catheters. Proximally located on body 18 is suction connector 20 attachable to a drainage receptacle or drainage tubing line. Fitted into body 18 is On-Off handle 21 shown in its normal on or open mode. Syringe port 22 has entrance fitting 23 which locks in a resilient silicone molded cup-shaped seal 24 which has a slit 25 in its upper surface which slit is normally biased to a closed position. The seal 24 is thus incorporated as part of body 18.

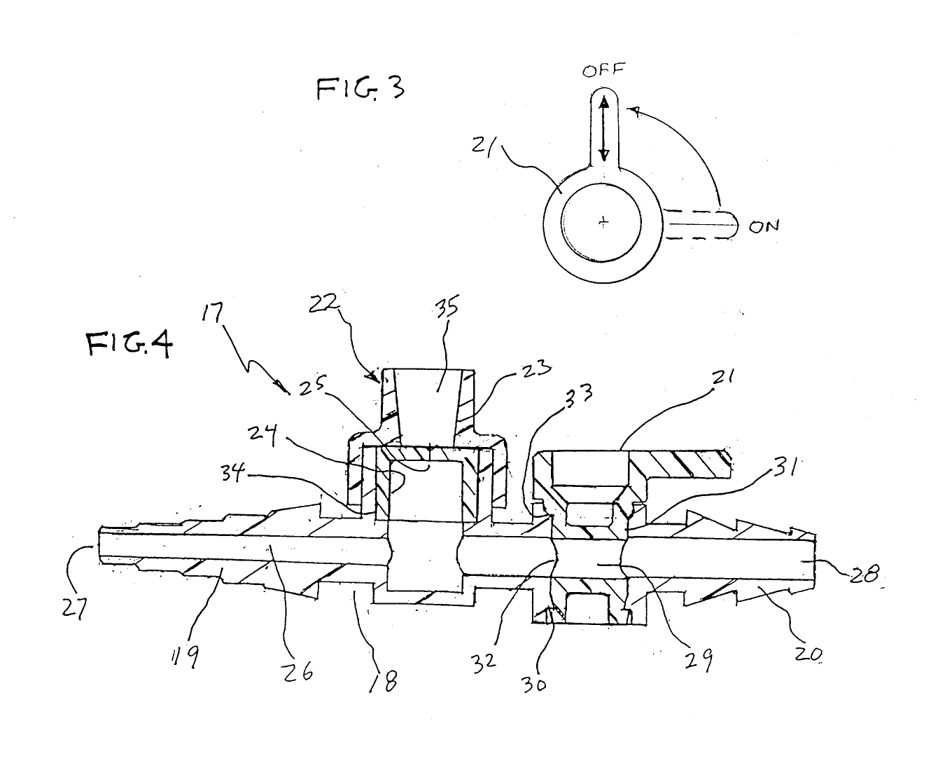

FIG. 3 shows On-Off handle 21 being manually turned from its normally fixed first on position to its second fixed off position. Handle 21 is keyed into body 18 such that there are built in stops which perfectly align the handle 90 degrees to either its simple on or off position which is easily comprehended by the user.

FIG. 4 is a cross sectional view of connector valve 17 wherein handle 21 is positioned to its first fixed on position. Body 18 has molded in linear central passageway 26 with inlet distal opening 27 and proximal exit 28 which forms a fluid communication path between distal opening 27 and proximal exit 28. Opening 27 permits fluid and air communication with any tube or catheter and exit 28 permits fluid and air to flow directly and unobstructively through passageway 26 and out to a drainage receptacle.

Proximal connector 20 similar in design to distal connector 19 will attach to any suction tubing and to any source of suction or to gravity drainage or drainage bag or receptacle.

Handle 21 has one large lumen cross-bore 29 and molded in undercut 30 which snap fits handle 21 into body 18.

Handle 21 has internal barrel 31 which forms a close tolerance fit with cross passage 32 within body 18. Undercut 30 forms a downward compression fit at tapered wedge 33 on handle 21 to form a liquid and airtight seal between handle 21 and body 18.

Handle 21 will rotate barrel 31 90 degrees within passage 32 to either open or close off passageway 26 from exit 28 as an On-Off shut off means. When handle 21 is positioned to its first fixed on position, there is a free unencumbered flow of fluid or air through passageway 26 from distal opening 27 right on through out exit 28.

When handle 21 is positioned or turned to its second fixed off position as shown in FIG. 3, then fluid and air flowing through passageway 26 is blocked from exit 28.

Formed as part of body 18 is upright collar 34 into which is fitted silicone molded slit seal 24. Permanently locking in slit seal 24 is entrance fitting 23 which is molded from polycarbonate. Slit seal 24 and entrance fitting 23 now form syringe port 22. Fitting 23 is sonic welded or solvent bonded to collar 34. Fitting 23 slightly compresses downward on top of seal 24 to form a liquid tight seal between fitting 23 and seal 24, and this downward compression also forces slit 25 in a normally biased liquid tight inward closed position. FIG. 4 thus depicts seal 24 in its normally biased sealed closed to atmosphere seal.

Fitting 23 has tapered friction fit opening 35 which will accept any piston or irrigation syringe tip. Collar 34 retains slit seal 24 such that slit 25 is normally biased closed to prevent any loss of fluid or air out slit seal 24. In normal operation, the closed system connector valve 17 is directly attached to any catheter or tube by distal connector 19 and also attached proximally to a tubing line by connector 20. Once connected, the connector valve 17 forms a closed system device such that fluid or air cannot escape from the valve.

When suction is applied and the valve is normally positioned to its first fixed open position, fluid or air will flow freely through passageway 26 and into a drainage receptacle. If the patient needs to be disconnected from the suction line and wants to become ambulatory, then the handle is turned to its second fixed off position and the system remains closed. Patient can now ambulate with the connector valve as part of the indwelling tube without fear of spillage. Reconnection to the suction line can take place at any future time. As such, the system remains closed at all times.

Often liquid or dissolved medications may want to be administered without having to disconnect closed connector valve 17 from the catheter or suction line by using a 60 cc catheter tip piston or irrigation syringe.

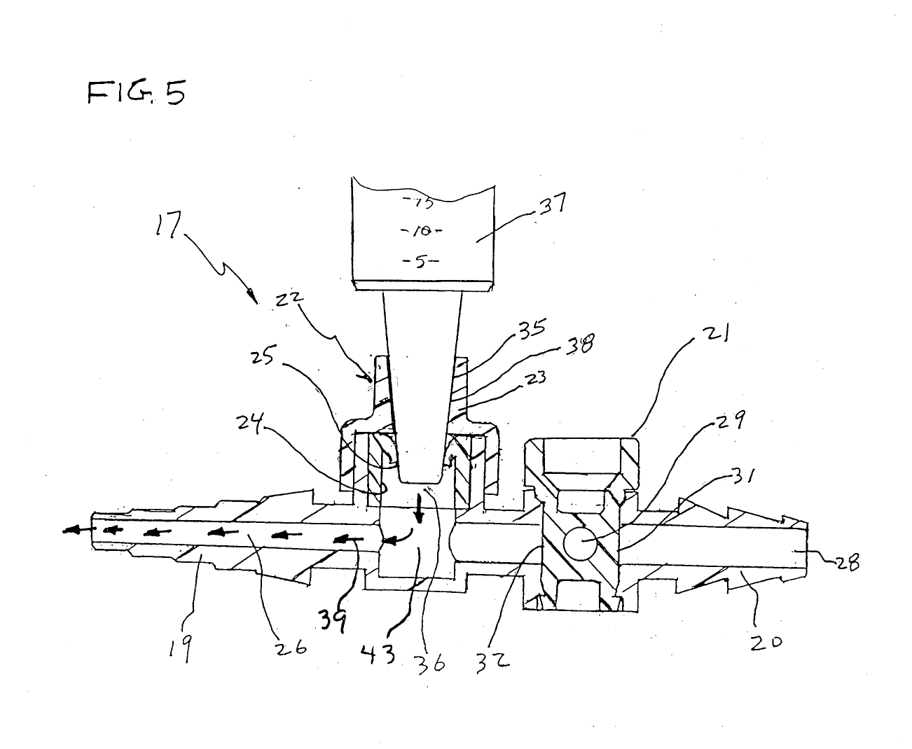

FIG. 5 depicts piston syringe 37 having syringe tip 36 opening slit seal 24 by direct insertion of syringe tip 36 into slit seal 24. Fitting 23 having friction fit opening 35 is so dimensioned to provide a friction fit with syringe nozzle 38 such that tip 36 will slightly open slit 25 on silicone slit seal 24. This is so syringe nozzle 38 will form a rigid solid friction fit with tapered opening 35. This fit limits the insertion of nozzle 38 such that tip 36 will not overstretch or overpower slit seal 24 to insure repeated use of syringe port 22 without failure.

In order to accommodate the large taper of 60 cc catheter tip syringe nozzle 38, silicone seal 24 must be about 0.500 inches in diameter and slit 25 must be at least 0.325 inches in length. However, syringe port 22 can easily be designed to accommodate the smaller nozzles on luer tip syringes by making silicone seal 24 smaller in diameter and having slit 25 shorter in length. Also in a luer tip syringe design, entrance fitting 23 can have a flat flush with the silicone seal 24 top design or a shorter in height opening 35. The cylindrical hat-shaped configuration of the seal enhances moldability as well as placement into the fitting 23; however, alternate shaped seals could be used, e.g., a simple flat disc shape. Also, the directional orientation of the slit 25 with respect to the fitting 23 can be modified from that shown.

When medication delivery is desired, handle 21 is turned to its off position which closes off cross bore 29 and prevents and blocks fluid or air flow out exit 28 as shown in FIG. 5.

Medication can now be infused directly in front of handle 21 and directly into passageway 26 in front of handle 21. The function of syringe port 22 and handle 21 are completely separate and not dependent on each other for function.

Slit seal 24 will automatically open and close shut by insertion and removal of syringe tip 36 to maintain a closed system at all times. Further, since syringe fluid 39 flows directly into passageway 26 there is no clogging of handle 21 or cross bore 29 with undissolved medication.

Reduced clogging in passageway 26 is enhanced by incorporation of a fluid-mixing chamber 43 directly downstream of syringe tip 36. Syringe tip 36 fluid flows into the relatively large diameter fluid mixing chamber 43 to create a turbulent swirling action in chamber 43 to keep crushed and dissolved granular medication in suspension and flowing freely into passageway 26.

Once syringe 37 is removed then handle 21 can return to its normal first fixed open position as depicted in FIG. 4 while the entire closed system connector valve 17 remains closed to atmosphere to all times. Since slit seal 24 functions automatically, there is no fear of splashing or splattering of stomach contents. Also, there is no need for a separate cap for syringe port 22 or a tethered cap for proximal connector 20.

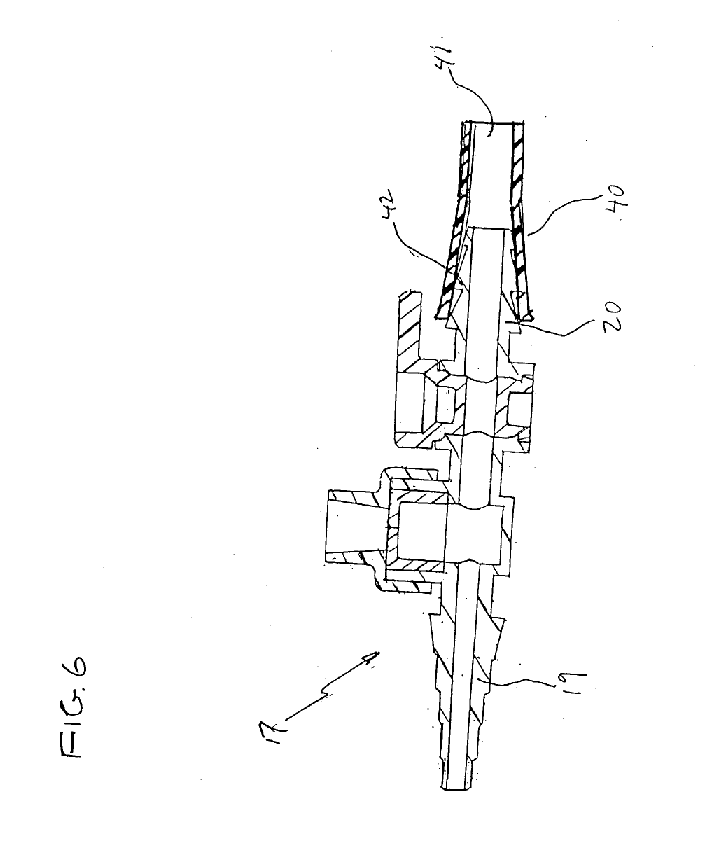

FIG. 6 shows the addition of PVC or silicone resilient adapter 40 to proximal connector 20 and forms a grip fit with barbed edges 42 on connector 20. Opening 41 on adapter 40 will now accept any enteral delivery set whether pump or gravity as well as any bolus syringe. Frontal connector 19 will attach directly to a wide variety of any size indwelling gastrostomy tubes due to its gradually increasing diameter steps.

Adapter 40 can be removed and the connector valve 17 returns to its original usage if desired.

FIGS. 7, 8, and 9 are top views of connector 17 to be used in conjunction with the following Directions For Use to make the directions easily understood.

FIG. 7 shows catheter connector end 44, feeding or patient tube 45, feeding set 46, flexible adapter 47, and handle 48 wherein connector valve 17 is shown in its feeding procedure mode.

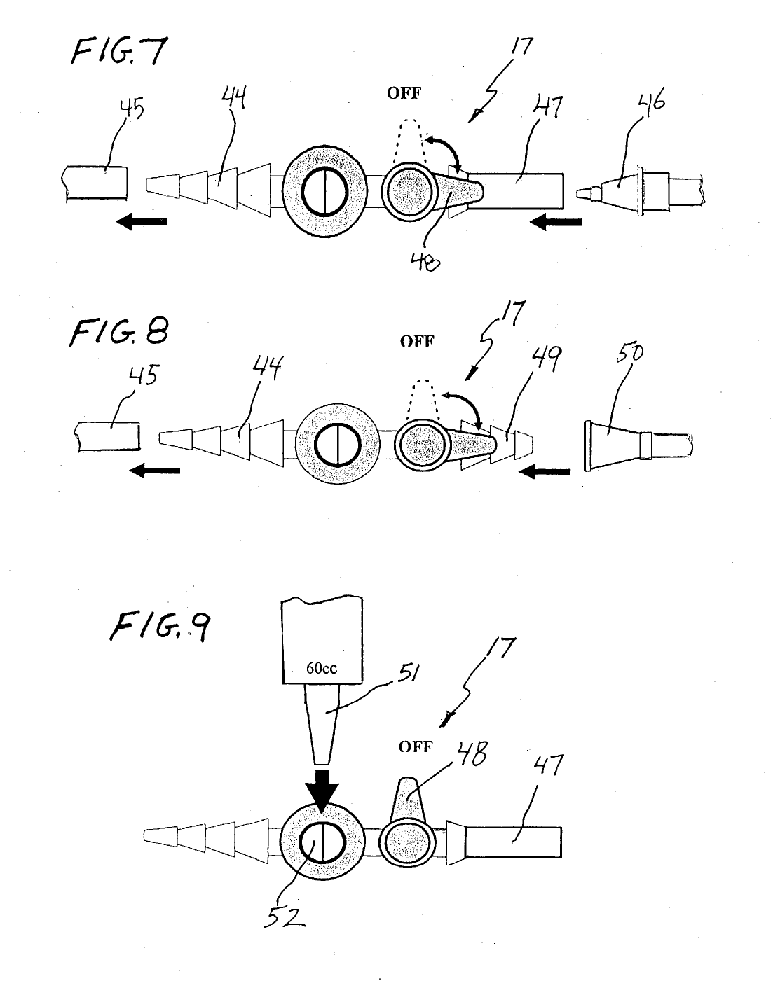

FIG. 8 shows flexible adapter 47 removed from proximal end 49 and end 49 connectable to suction tubing 50 wherein connector valve 17 is shown in its drainage and suction procedure mode.

FIG. 9 shows handle 48 turned 90° to its closed position and catheter syringe tip 51 ready to be inserted into syringe port seal 52 wherein connector valve 17 is shown in its irrigation/lavage and medication delivery procedure mode.

Directions for Use

Feeding Procedure (See FIG. 7)

Connector Valve 17 Replaces Existing Open Style Feeding Adapters

1. Attach catheter connector end to feeding tube.

2. Insert feeding set or bolus syringe tip into flexible adapter.

3. Turn handle to the ON position to deliver feeding formula.

4. Turn handle to the OFF position when feeding is complete, and remove feeding set from flexible adapter.

Drainage and Suction Procedure (See FIG. 8)

Connector Valve 17 Replaces 5-in-1 Connectors

1. Attach catheter connector end to patient tube.

2. Remove flexible adapter and attach suction tubing directly to connector valve.

3. Turn handle to the ON position to suction or drain.

4. Turn handle to the OFF position and remove suction tubing.

Irrigation/Lavage and Medication Delivery Procedure (See FIG. 9)

1. Turn handle to the OFF position.

2. Hold the connector valve securely and push catheter tip syringe firmly into syringe port seal until tight.

3. Dispense fluid or medication into the connector valve.

4. Remove syringe. The syringe port seal will close automatically.

To ambulate patient, turn handle to OFF position and disconnect feeding set or suction tubing. Patient can then ambulate as desired. Feeding and drainage can be resumed as needed by repeating above procedures. The connector valve remains as part of catheter according to accepted medical standards.

As can be seen, the present invention overcomes many of the objections of the prior art and provides a truly closed system connector valve for feeding, drainage and suction, and the infusion of irrigation/lavage and medication delivery.

Many variations in design, materials, and configurations can take place without departing from the broad scope of the underlying invention.

I claim:

1. A closed system connector comprising: a body having a passageway therein extending between a distal opening and a proximal exit within the body; an On-Off valve positioned adjacent to the proximal exit and manually operable to a first normally open position permitting fluid communication from the distal opening to the proximal exit and said valve further manually operable to a second closed position preventing fluid communication from the distal opening to the proximal exit; a syringe port positioned adjacent to the distal opening, the port having a seal normally biased to a sealed closed to atmosphere position, said seal directly engageable with the tip of a syringe thus permitting the seal to open to infuse medication from the syringe into the body passageway when the On-Off valve is positioned to its second closed position, the syringe port automatically returning to its normally biased sealed closed position upon removal of the syringe tip from the seal.

2. The closed system connector of claim 1 wherein the On-Off valve has fixed stops.

3. The closed system connector of claim 1 wherein the seal is a molded resilient component.

4. The closed system connector of claim 1 wherein the distal opening is connectable to a patient tube.

5. The closed system connector of claim 1 wherein the proximal exit is connectable to a drainage or suction source.

6. The closed system connector of claim 1 wherein the proximal exit includes a flexible adapter.

7. A system for delivering medication into a patient comprising: a closed system connector having a distal opening and a proximal exit, the distal opening adapted for attachment to an indwelling patient tube, the proximal exit adapted for attachment to a drainage receptacle, the connector having a fluid flow passageway permitting fluid flow between the distal opening and the proximal exit, the connector including an On-Off valve manually operable to a first open position permitting fluid flow communication from the distal opening to the proximal exit and the valve further manually operable to a second closed position preventing fluid flow communication from the distal opening to the proximal exit, the connector also having a syringe port, said syringe port having a normally biased sealed closed to atmosphere seal, said seal adapted for being opened by the direct insertion of a syringe tip into the seal to infuse medication into the connector and on into the patient when the On-Off valve is positioned to its second closed position, said seal automatically returning to be a normally biased sealed closed to atmosphere sealed position upon removal of the syringe tip from the seal.

8. The closed system connector of claim 7 wherein the On-Off valve has fixed stops.

9. The closed system connector of claim 7 wherein the distal opening is connectable to a patient tube.

10. The closed system connector of claim 7 wherein the proximal exit is connectable to a drainage or suction source.

11. The closed system connector of claim 7 wherein the proximal exit includes a flexible adapter.

12. The closed system connector of claim 7 wherein the seal is a resiliently molded component.

13. The closed system connector of claim 12 wherein the seal includes a cylindrical body and a closed top wall, said top wall having a slit therethrough, said seal body positioned in said syringe port.

14. The method of providing drainage and infusion to a patient who is connected to an indwelling tube comprising the steps of: attaching an inlet opening of a closed system connector device to the indwelling tube and attaching the exit opening of such connector to a drainage tubing line; such that the connector provides drainage to flow from the indwelling tube through the connector and out to the drainage tubing line when an On-Off valve which is part of the connector is normally positioned in its open position; providing syringe infusion into the connector and thus into the indwelling tube by manually positioning the On-Off valve to the off position which prevents drainage to flow through the connector; providing a normally sealed closed syringe port seal as part of the connector wherein the syringe port seal is opened by the direct insertion of the syringe tip to deliver medication infusion into the indwelling tube when the On-Off valve is positioned in the off position; thereafter removing the syringe tip from the syringe port seal after medication infusion is completed wherein the syringe port seal automatically returns to its normally sealed closed position upon removal of the syringe tip from the syringe port seal; and manually returning the On-Off valve to its normally on position to restore drainage flow from the indwelling tube, through the connector, and out the drainage tubing line.

15. The method of claim 14 wherein the On-Off valve positions between fixed stops.

16. A closed system medical connector adapted to be attached at its distal end to a medical tube and at its proximal end to an infusion delivery device comprising: a body member having a passageway therein extending between its distal end and its proximal end; the passageway permitting fluid flow between the proximal end and the distal end when the distal end is attached to an infusion delivery device, an On-Off fluid controller as part of the body member located adjacent to the proximal end permitting unobstructed fluid flow between the proximal end and the distal end when the fluid controller is positioned in the on position and the controller fully obstructing fluid flow between the proximal end and the distal end when the controller is positioned in the off position, said body member including a syringe port seal located adjacent to the distal end, the syringe port seal normally biased to a sealed closed to atmosphere position and the seal directly engageable with the tip of a syringe thus permitting the seal to open to infuse medication into the passageway and solely out the distal end when the fluid controller is positioned in the off position and the syringe port seal automatically returning to be normally biased sealed closed position upon removal of the syringe tip from the syringe port seal.

17. The system of claim 16 wherein the controller has an On-Off handle.
